SEQUENCE LISTING (1 sequence)

SEQ ID NO: 1 - protein, 10 aa, Artificial
  PEPTIDE (1)..(10): Peptide SynB3  peptide derived from the protegrin family
RRLSYSRRRF

(19) United States
(12) Patent Application Publication
(10) Pub. No.: US 2004/0248806 A1
(43) Pub. Date: Dec. 9, 2004

(54) Compounds comprising an analgesic molecule linked to a vector that can vectorise said molecule through the hematoencephalic barrier and pharmaceutical compositions containing same

(76) Inventors: James Temsamani, Nimes (FR); Philippe Clair, Nimes (FR); Anthony R Rees, Saint-Chaptes (FR)

Correspondence Address: BACHMAN & LAPOINTE, P.C., 900 CHAPEL STREET, SUITE 1201, NEW HAVEN, CT, 06510, US

(21) Appl. No.: 10/468,412

(22) PCT Filed: Feb. 22, 2002
(86) PCT No.: PCT/FR02/00667

(30) Foreign Application Priority Data
Feb. 23, 2001 (FR) 01/02504

(51) Int. Cl. A61K 38/08; C07K 7/08

(52) U.S. Cl. 514/13; 514/14; 530/326; 530/327

(57) ABSTRACT

The invention relates to compounds comprising an analgesic molecule which is selected from morphine and the derivatives and metabolites thereof and which is vectorised by means of its link to a vector such that the analgesic molecule passes through the hematoencephalic barrier. The invention also relates to the use of the compounds for the preparation of medicaments that are used to treat pain.

### BACKGROUND OF THE INVENTION

The present invention relates to compounds consisting of an analgesic molecule vectorized through being linked to a vector such that said analgesic molecule crosses the blood-brain barrier, and also to the use of said compounds for preparing medicinal products which are of use for treating pain.

Morphine constitutes one of the compounds most commonly used in the treatment of medium and high strength pain. Unfortunately, treatment with morphine is often accompanied by adverse effects such as: euphoria or drowsiness, respiratory depression, inhibition of intestinal transit, nausea, vomiting and, especially, a syndrome of addiction and induction of tolerance (Cherny et al., 1996). After it is administered to animals or to the patient, morphine undergoes an important first-pass effect in the liver, the consequence of which is a low and variable bioavailability depending on the route of administration. Morphine undergoes mainly an enantioselective glucuronidation catalyzed by the enzyme UDP-glucuronyltransferase (UGT), and the liver appears to be the main site for its bioconversion. One of the main derivatives of morphine is its metabolite, morphine-6-glucuronide (M6G).

This metabolite also possesses analgesic activity. Ligand-opiate receptor binding studies carried out in vitro have shown that M6G binds to opioid receptors and that it has 3 to 5 times less affinity for μreceptors than morphine (Christensen & Jorgensen 1987; Frances et al., 1992). There are two types of μ receptor: μ1 receptors, which are very high affinity and low capacity receptors, and μ2 receptors, which are low affinity and high capacity receptors (Pasternak & Wood, 1986). Binding to μ1 receptors causes an analgesic reaction of the supraspinal type and a decrease in acetylcholine turnover, whereas binding to μ2 receptors causes an analgesic reaction of the spinal type and is responsible for respiratory depression, inhibition of intestinal transit and several signs associated with addiction.

Morphine and its active metabolite, M6G, must cross the blood-brain barrier (BBB) before being distributed in the brain, the main site of action of their analgesic effects. Morphine is a base with a pKa of between 8 and 9, and therefore weakly ionized at blood pH, the penetration of which into the brain has been described as a simple diffusion. Its metabolite, M6G, is an acid (pKa between 2 and 3) which is highly ionized at blood pH. Because of its hydrophilicity, penetration of M6G into the brain is very limited.

It has been demonstrated that intracerebroventricular administration, at equal dose, of morphine and of M6G produces an analgesic effect which is 50 to 100 times greater for M6G than for morphine (Pasternak et al., 1987; Frances et al., 1982; Stain et al., 1995). On the other hand, when administered by systemic injection, the two molecules have roughly the same analgesic activity. These results clearly indicate that the penetration of M6G into the brain is very limited compared to that of morphine.

The blood-brain barrier consists of endothelial cells which form an obstacle, in various ways, to molecules which attempt to cross them. They constitute a physical barrier represented by the tight junctions which bind to one another and prevent anything from passing via the paracellular route, all the more so since the endocytotic activity therein is low. All this greatly limits the passage of molecules from the plasma to the extracellular space in the brain.

Thus, in the context of its research studies, the applicant has demonstrated that vectors, such as linear peptides derived from natural peptides such as protegrin and tachyplesin, transport active molecules across the BBB. Protegrin and tachyplesin are natural peptides, the structure of which is of the hairpin maintained by disulfide bridges type. These bridges play an important role in the cytolytic activity observed on human cells. Irreversible reduction of these bridges makes it possible to obtain linear, noncytotoxic peptides having the ability to rapidly cross mammalian cell membranes via a passive mechanism which does not use a membrane-bound receptor.

The studies and results concerning these linear peptides and their use as vectors for active molecules across the blood-brain barrier have been described in French patent applications No. 98/15074, filed on Nov. 30, 1998, and No. 99/02938, filed on Nov. 26, 1999, by the applicant.

### SUMMARY OF THE INVENTION

A subject of the present invention is compounds consisting of an analgesic molecule linked to a vector that can vectorize said analgesic molecule across the blood-brain barrier.

Preferably, the vector that can vectorize said analgesic molecule across the blood-brain barrier is a linear peptide derived from the protegrin or tachyplesin family.

The expression “peptide derived from the protegrin family” is intended to mean any peptide which corresponds to formula (I) below:

BXXBXXXXBBBXXXXXXB (I)

and the expression “peptide derived from the tachyplesin family” is intended to mean any peptide which corresponds to formula (II) below:

BXXXBXXXBXXXXBBXB (II)

in which:

the groups B, which may be identical or different, represent an amino acid residue the side chain of which carries a basic group, and

the groups X, which may be identical or different, represent an aliphatic or aromatic amino acid residue,

or said peptides of formula (I) or (II), in retro form, consisting of amino acids in the D and/or L configuration,

or a fragment thereof consisting of a sequence of at least 5, and preferably of at least 7, successive amino acids of the peptides of formula (I) or (II).

The following meanings for B and X may be mentioned by way of example:

B is chosen from arginine, lysine, diaminoacetic acid, diaminobutyric acid, diaminopropionic acid and ornithine.

X is chosen from glycine, alanine, valine, norleucine, isoleucine, leucine, cysteine, cysteine^{Acm}, penicillamine, methionine, serine, threonine, asparagine, glutamine, phenylalanine, histidine, tryptophan, tyrosine, proline, Abu, amino-1-cyclohexanecarboxylic acid, Aib, 2-aminotetralincarboxylic, 4-bromophenylalanine, tert-leucine, 4-chlorophenylalanine, beta-cyclohexylalanine, 3,4-dichlorophenylalanine, 4-fluorophenylalanine, homoleucine, beta-homoleucine, homophenylalanine, 4-methylphenylalanine, 1-naphthylalanine, 2-naphthylalanine, 4-nitrophenylalanine, 3-nitrotyrosine, norvaline, phenylglycine, 3-pyridylalanine and [2-thienyl]alanine.

By way of nonlimiting example of an analgesic molecule, the invention envisions compounds chosen from opioids, such as encephalin or morphine, nonsteroidal anti-inflammatory compounds (NSAIDs), Cox 2-inhibiting compounds, NMDA receptor agonist compounds, calcium channel-blocking compounds, and neuropeptides, and in particular morphine derivatives. By way of example of morphine derivatives, mention may be made of those which have analgesic activity but which, as such, do not cross the blood-brain barrier, such as morphine metabolites, and in particular M6G.

Preferably, the analgesic molecule used in the context of the present invention is morphine, one of its derivatives or one of its metabolites. Most preferably, such a metabolite is M6G.

The sites for linking the analgesic molecule may be at the N-terminal or C-terminal end or else on the side chains of the vector peptide.

The functional groups such as —OH, —SH, —COOH or —NH₂ may be naturally present or can be introduced, either onto the vector or onto the analgesic molecule, or onto both.

The linking of the vector to the analgesic molecule can be carried out by any means of linkage which is acceptable given the chemical nature and the hindrance both of the vector and of the analgesic molecule. The linkages may be covalent, hydrophobic or ionic, and cleavable or noncleavable in physiological media or inside the cell.

This linking of the vector to the analgesic molecule can be carried out directly or indirectly.

When the linking is carried out indirectly, a linking agent may advantageously be used. By way of nonlimiting example of linking agents which can be used in the context of the invention, mention may be made of bi- or multifunctional agents containing alkyl, aryl, aralkyl or peptide groups, alkyl, aryl or aralkyl acids, aldehydes or esters, anhydride, sulfhydryl or carboxyl groups such as derivatives of maleymil benzoic acid or of maleymil propionic acid and succinimidyl derivatives, groups derived from cyanogen bromide or chloride, carbonyldiimidazole, succinimide esters or sulfonyl halides.

Advantageously, linkages involving at least one disulfide bridge are used, which linkages are characterized by their stability in the plasma after injection of the compound, and then, once the compounds of the invention have crossed the blood-brain barrier, said disulfide bridge is reduced, releasing the active analgesic molecule. The linking can be carried out at any site on the vector.

The analgesic compound vectorized consists of a morphine derivative coupled, via its 6-position, to the vector.

Most preferably, when the analgesic molecule is morphine, the vector is attached at the 6-position of said morphine molecule.

Most preferably again, when the analgesic molecule is morphine-6-glucuronide, the vector is attached at the carboxylic acid of the glucuronide residue of said morphine-6-glucuronide molecule.

A subject of the present invention is also the use of said vectorized compounds of an analgesic molecule, in a pharmaceutical composition, for preparing a medicinal product which is of use for treating pain.

Preferably, the pharmaceutical composition is in a form suitable for systemic, parenteral, oral, rectal, nasal, transdermal or pulmonary administration.

A subject of the invention is also a method for treating pain, consisting in administering to a patient a pharmaceutical composition comprising at least one vectorized compound consisting of an analgesic molecule linked to a vector, said peptide being a derivative of the protegrin or tachyplesin family.

### BRIEF DESCRIPTION OF THE DRAWINGS

The figures given in the appendix illustrate various results obtained by the applicant following experimental studies which enabled it to obtain the vectorized compounds of the invention:

FIG. 1 represents diagrammatically the chemical synthesis of a vectorized compound of morphine-6-glucuronide (M6G), a morphine metabolite;

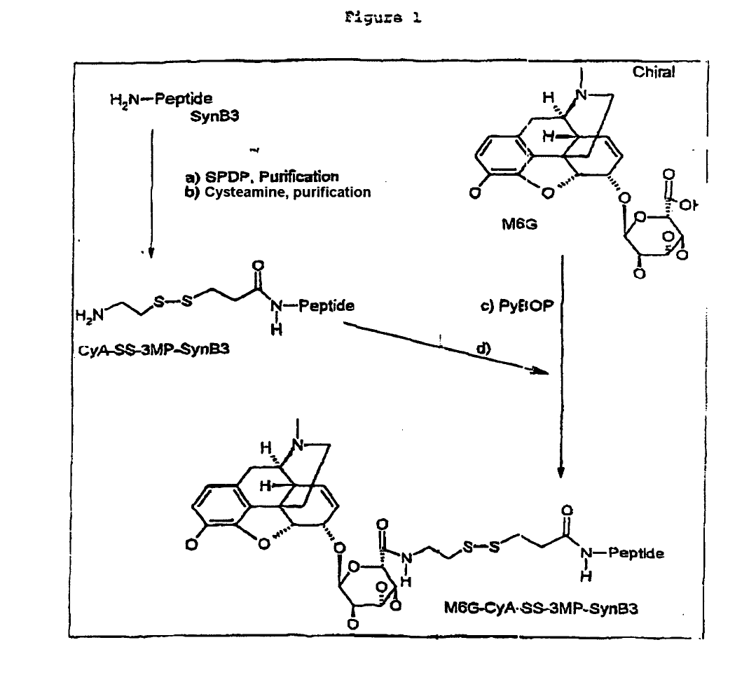

FIG. 2 illustrates the results of a comparative study of analgesic efficacy between compound 1 (morphine) and compound 2 (vectorized M6G);

FIG. 3 illustrates the results of a comparative study of analgesic efficacy between compound 1 (morphine), compound 2 (vectorized M6G) and compound 3 (free M6G);

FIG. 4 illustrates a comparative study of penetration into the BBB of free M6G (compound 1) with that of the vectorized M6G (compound 2);

FIG. 5 illustrates the results of a comparative study of the effect of morphine, of the vectorized M6G and of free M6G on respiratory depression. The compounds were used at the ED50 dose (FIG. 5, A), at a dose of 5×ED50 (FIG. 5, B) and at a dose of 10×ED50 (FIG. 5, C).

### DETAILED DESCRIPTION OF THE PREFERRED EMBODIMENT(S)

The present invention will be understood more clearly on reading the description of the experimental studies performed in the context of the research carried out by the applicant, which should not be interpreted as being limiting in nature.

I. Preparation of the Test Compounds

I.1. Chemical Synthesis of the Vectorized M6G

a) Synthesis of the Vector Peptide

The peptide SynB3 is assembled on solid phase according to a Foc/tu strategy, cleaved and deprotected with trifluoroacetic acid, and then purified by preparative reverse-phase high pressure chromatography and lyophilized. Its purity (>95%) and its identity are confirmed by analytical HPLC and mass spectrometry.

b) Coupling of the CyA-3 MP Link to the Vector Peptide

The peptide SynB3 of sequence RRLSYSRRRF (SEQ ID No. 1 in the sequence listing in the appendix) (one molar equivalent) is incubated for 30 minutes with one molar equivalent of the reagent SPDP (N-succinimidyl 3-(2-pyridyldithio)propionate in the solvent DMF (dimethylformamide) in the presence of two molar equivalents of DIEA (diisopropylethylamine). The resulting peptide, PySS-3 MP-SynB3, is precipitated with ether, and then purified by preparative reverse-phase high pressure chromatography and lyophilized. Its purity (>95%) and its identity are confirmed by analytical HPLC and by mass spectrometry.

One molar equivalent of peptide Py-SS-3 MP-SynB3 is incubated for 30 minutes with four molar equivalents of CyA, HCl (cysteamine hydrochloride) in the solvent DMF (dimethylformamide) in the presence of four molar equivalents of DIEA. The resulting peptide, CyA-SS-3 MP-SynB3, is precipitated with ether, and then purified by preparative reverse-phase high pressure chromatography and lyophilized. Its purity (>95%) and its identity are confirmed by analytical HPLC and by mass spectrometry.

c) Coupling of M6G to CyA-SS-3 MP-SynB3

One molar equivalent of M6G is incubated with two molar equivalents of PyBOP, in the presence of four molar equivalents of DIEA in the solvent DMF.

One molar equivalent of peptide CyA-SS-3 MP-SynB3 dissolved in DMF is then added to the reaction mixture, and then incubated for 30 minutes. The product formed, M6G-CyA-SS-3 MP-SynB3, is precipitated with ether, and then purified by preparative reverse-phase high pressure chromatography and lyophilized. Its purity (>95%) and its identity are confirmed by analytical HPLC and by mass spectrometry.

I.2. The Test Compounds

Table 1 below recapitulates the various compounds tested.

TABLE 1

| Compound | |
|---|---|
| Compound 1 | M6G |
| Compound 2 | M6G-S-S-RRLSYSRRRF |
| Compound 3 | Morphine |

II. Comparison of the Analgesic Effect

II.1. Assay Used: Hot Plate Test

a) Experimental Conditions

The hot plate test is carried out according to the experimental protocol described by Eddy N B et al., Synthetic analgesics, 1—Methadone isomers and derivatives, J. Pharmacol. Exp. Ther. 1950 (98): 121-137.

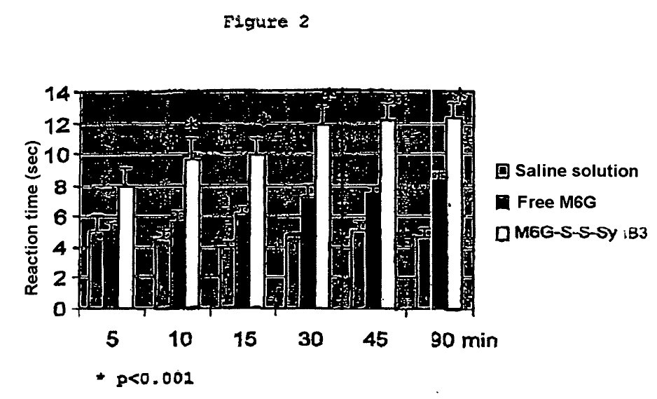

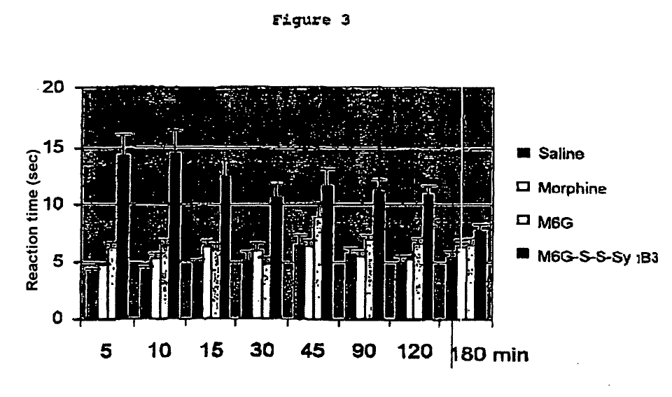

The mouse placed on a plate heated at 55° C. shows the pain it is experiencing by licking its front feet or, more rarely, by jumping. The reaction time is then noted. The compounds studied are administered intravenously into the caudal vein of the mouse at a dose of 1 mg/kg. The reaction time is then measured after 5 to 90 minutes (results given in FIG. 2) and from 5 to 180 minutes (results given in FIG. 3).

b) Results

Initially, the inventors compared the analgesic activity of the free M6G compound with that of the compound obtained by vectorizing the M6G with the peptide SynB3. The results obtained are represented in FIG. 2 and clearly show that the analgesic effect of the vectorized M6G (compound 2) is much more significant than that obtained with the free M6G (compound 1). This analgesic effect is slow and lasting, even after 90 minutes post-administration.

In another experiment, the effect of the vectorized M6G (compound 2) was compared with that of morphine (compound 3) at the same dose of 1 mg/kg. The results from this experiment (FIG. 3) clearly show that the vectorized M6G (compound 2) has a much greater analgesic effect than that of morphine, and at a time ranging up to 120 minutes post-administration.

II.2. Assay Used: Tail Flick Test

a) Experimental Conditions

The mouse tail is placed in front of an infrared source. The light is focused on the ventral surface of the tail so as to produce a surface temperature of 55° C. As soon as the mouse moves the tail, the reaction time is then measured. The compounds studied are administered subcutaneously.

Three measurements are taken before administration of the product so as to have a baseline time. The percentage of mice in which analgesia has occurred is then represented by the number of mice having a reaction time which is at least double the baseline time divided by the total number of mice. The ED50 dose represents the concentration which gives 50% of mice in which analgesia has occurred.

b) Results

Firstly, we compared the analgesic effect of the morphine or free M6G compounds with respect to that of the compound obtained by vectorizing the M6G with the peptide SynB3, using two routes of administration: intravenous and subcutaneous. We determined the ED50, which represents the dose which gives an analgesic effect in 50% of the mice in the “tail flick” model, for each product. Table 2 shows that the dose required to induce an analgesic effect in 50% of mice was much lower for the vectorized M6G (compound 2) than for morphine (compound 3) or the free M6G (compound 1). This clearly indicates that the analgesic effect of the vectorized M6G is much more significant than that of the other products tested.

TABLE 2
Comparison of the analgesic activity

| | | | | Vectorized |
|---|---|---|---|---|
| | Route of | Morphine | M6G | M6G |
| | administration | (μmol/kg) | (μmol/kg) | (μmol/kg) |
| ED50 | subcutaneous | 11.6 | 6.6 | 1.7 |
| | intravenous | 15.4 | >5.7 | 1.08 |

Secondly, we measured the duration of the analgesic effect in these mice. Table 3 shows that, not only does the vectorized M6G have a more analgesic effect, but this effect lasts longer than that of the M6G or of the morphine.

TABLE 3
Comparison of the analgesic effect time

| | Route of | | | Vectorized |
|---|---|---|---|---|
| | administration | Morphine | M6G | M6G |
| Duration of the | subcutaneous | 90 min | 150 min | 300 min |
| effect | intravenous | 60 min | 90 min | 180 min |

III. Comparison of the Receptor Affinity

a) Experimental Conditions

Tissue homogenates are prepared from calf brains (thalamus for μ1 and μ2 and frontal cortex for delta). [³H][D-Ala²,D-Leu⁵]enkephalin (DADLE) (0.7 nM) is incubated with 3 ml of tissue homogenate (15 mg of tissue per ml) in the presence of 10 nM of [D-Pen²,D-Pen⁵]enkephalin (DPDPE) and of increasing concentrations of free or vectorized M6G. Under these conditions, specific binding to μ1 binding sites is observed. For the μ2 receptor, [D-Ala², MePhe⁴,Gly(ol)⁵]enkephalin (DAMGO) (1 nM) is incubated with 3 ml of tissue homogenate in the presence of 5 nM [D-Ser2,Leu⁵]enkephalin-Thr⁶ (DSLET) and of increasing concentrations of free or vectorized M6G. For the delta receptor, the tissue is incubated with [³H] [D-Pen²,D-Pen⁵]enkephalin in the presence of free or vectorized M6G. The nonspecific binding is determined by adding to the labeled ligands 1 μM of levallorphan.

b) Results

M6G is an active metabolite of morphine which binds to μ receptors with very high affinity. We compared the affinity of free M6G (compound 1) with that of the vectorized M6G (compound 2) for μ1, μ2 and delta receptors.

The results show that adding a linker and a vector peptide (SynB3) to M6G increases its affinity for the μ1 receptor by approximately 3-fold and its affinity for the μ2 receptor by approximately 10-fold. On the other hand, the binding to the delta receptor remained the same.

TABLE 4
Affinity for μ1 and μ2 receptors
(Ki expressed in nM)

| Compound | μ1 | μ2 | delta |
|---|---|---|---|
| M6G (compound 1) | 2.67 | 5.82 | 23 |
| M6G-S-S-SynB3 | 0.95 | 0.60 | 19 |
| (compound 2) | | | |

IV. Comparison of the Penetration into the Brain

a) Experimental Conditions: In Situ Brain Perfusion

Mice (20-25 g, Iffa-Credo; l'Arbresle, France) are anesthetized. After exposure of the common carotid, the right external carotid artery is ligated at the level of the bifurcation with the internal carotid, and the common carotid is ligated between the heart and the site of the implantation of the catheter (polyethylene catheter, ID: 0.76). Said catheter, pre-filled with a heparin solution (100 units/ml) is inserted into the common carotid. The mice are perfused with a perfusion buffer (128 mM NaCl, 24 mM NaHCO₃, 4.2 mM KCl, 2.4 mM NaH₂PO₄, 1.5 mM CaCl₂, 0.9 mM MgSO₄ and 9 mM D-glucose). This buffer is filtered and then a mixture containing 95% O₂/5% CO₂ is bubbled through in order to maintain the pH in the region of 7.4 and to supply the brain with oxygen during the perfusion.

The mice are perfused with the buffer containing the free M6G (compound 1: specific activity 84 mCi/mg) or the vectorized M6G (compound 2; specific activity 14.3 mCi/mg). Just before the start of the perfusion, the heart is stopped by sectioning the ventricles, in order to avoid reflux of the perfusate during the perfusion. The right hemisphere is then perfused at a rate of 10 ml/min for 60 seconds, after which time the mouse is decapitated. The amount of radioactivity in the right hemisphere is then measured and the brain penetration index (Kin) is calculated.

b. Results

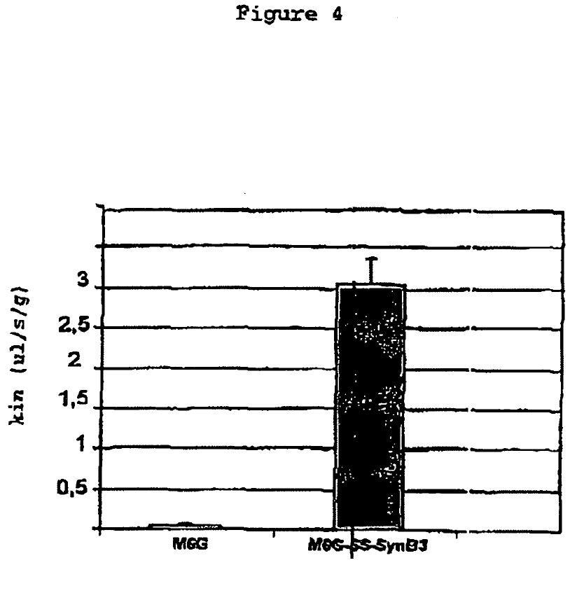

In this study, we compared the penetration through the BBB of free M6G (compound 1) with that of the vectorized M6G (compound 2). The two products were perfused into the brain of the mouse. After 60 seconds of perfusion in the buffer, the penetration of the products is estimated by the influx constant, or Kin, in μl/sec/g. FIG. 4 shows that vectorizing the M6G with the vector SynB3 increases its passage into the brain by approximately 100-fold after a perfusion of 60 seconds in buffer.

V. Comparison of Respiratory Depression

a. Experimental Conditions

The respiratory depression was calculated in rats. The product is injected into the animals subcutaneously and, after a certain amount of time, an aliquot of blood is taken from the femoral artery via a catheter implanted beforehand. During the study, the animals are placed in a calm place. After the blood sample has been taken, the oxygen saturation (SO₂) and the CO₂ pressure (PCO₂) are measured. The SO₂ values are measured in % O₂.

The rats were injected with 3 doses of each product, which correspond to ED50, 5×ED50 and 10×ED50 (see Table 5). After time periods ranging from 0 to 150 min (30, 60, 90, 120, 150 min), a blood sample is taken and the O₂ saturation (SO₂) and CO₂ pressure are measured.

TABLE 5
Doses used for the respiratory depression study

| | ED50 | 5 × ED50 | 10 × ED50 |
|---|---|---|---|
| | (μmol/kg) | (μmol/kg) | (μmol/kg) |
| Morphine | 13.1 | 66 | 131 |
| M6G | 8.7 | 43.7 | 87 |
| Vectorized M6G | 4.3 | 21.8 | 43 |

b. Results

Although morphine is the most commonly used substance in the treatment of medium and high strength pain, its use induces respiratory depression in patients. We therefore compared the effect of vectorized M6G with that of morphine and of free M6G.

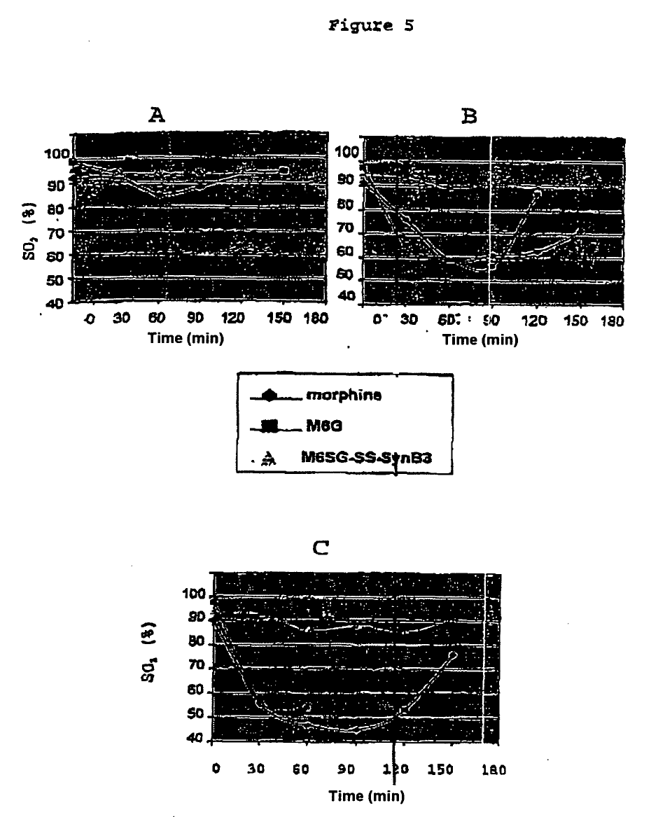

At the ED50 dose (FIG. 5, A), no effect was observed for the vectorized M6G or the M6G. We nevertheless noted a small decrease in O₂ saturation, for the morphine, between 30 and 90 min.

At the 5×ED50 and 10×ED50 doses (FIG. 5, B and C), the morphine and the M6G induced a very significant decrease in the oxygen saturation. The oxygen level fell to almost 50% between 30 and 90 min. On the other hand, for the vectorized M6G, no notable decrease was obtained.

At the 10×ED50 dose (FIG. 5, C), the large decrease observed with the free M6G caused the death of some animals at the 60 min time point.

The results given in FIG. 5 demonstrate that the vectorized M6G product not only has an analgesic effect which is better than free M6G or morphine, but it also makes it possible to significantly decrease the side effects associated with morphine.

1. A compound, characterized in that it consists of an analgesic molecule chosen from the group consisting of morphine, its derivatives and its metabolites, linked to a vector that can transport said analgesic molecule across the blood-brain barrier, said vector being a linear peptide corresponding to either of formulae (I) and (II) below:
BXXBXXXXBBBXXXXXXB (I)
BXXXBXXXBXXXXBBXB (II)
in which:
the groups B, which may be identical or different, represent an amino acid residue the side chain of which carries a basic group, and
the groups X, which may be identical or different, represent an aliphatic or aromatic amino acid residue,
or said peptides of formula (I) or (II), in retro form, consisting of amino acids in the D and/or L configuration,
or a fragment thereof consisting of a sequence of at least 5, and preferably of at least 7, successive amino acids of the peptides of formula (I) or (II).

2. The compound as claimed in claim 1, wherein the group B is chosen from arginine, lysine, diaminoacetic acid, diaminobutyric acid, diaminopropionic acid and ornithine.

3. The compound as claimed in claim 1, wherein the group X is chosen from glycine, alanine, valine, norleucine, isoleucine, leucine, cysteine, cysteine^{Acm}, penicillamine, methionine, serine, threonine, asparagine, glutamine, phenylalanine, histidine, tryptophan, tyrosine, proline, Abu, amino-1-cyclohexanecarboxylic acid, Aib, 2-aminotetralincarboxylic, 4-bromophenylalanine, tert-leucine, 4-chlorophenylalanine, beta-cyclohexylalanine, 3,4-dichlorophenylalanine, 4-fluorophenylalanine, homoleucine, beta-homoleucine, homophenylalanine, 4-methylphenylalanine, 1-naphthylalanine, 2-naphthylalanine, 4-nitrophenylalanine, 3-nitrotyrosine, norvaline, phenylglycine, 3-pyridylalanine and [2-thienyl]alanine.

4. The compound as claimed in claim 1, wherein the sites for linking the analgesic molecule to the vector are located at the N-terminal or C-terminal end or else on the side chains of said vector.

5. The compound as claimed in claim 1, wherein the linking of the analgesic molecule to the vector is carried out by means of a functional group which is naturally present or which is introduced either onto the vector or onto the analgesic molecule, or onto both.

6. The compound as claimed in claim 5, characterized in that the functional group is chosen from the groups: —OH, —SH, —COOH and —NH₂.

7. The compound as claimed in claim 1, wherein the linkage between the analgesic molecule and the vector is a linkage chosen from a covalent linkage, a hydrophobic linkage, an ionic linkage, and a linkage which is cleavable or a linkage which is noncleavable in physiological media or inside the cells.

8. The compound as claimed in claim 1, wherein the linking of the analgesic molecule to the vector is direct linking.

9. The compound as claimed in claim 1, wherein the linking of the analgesic molecule to the vector is indirect linking carried out by means of a linking agent.

10. The compound as claimed in claim 9, wherein the linking agent is chosen from bi- or multifunctional agents containing alkyl, aryl, aralkyl or peptide groups, alkyl, aryl or aralkyl acids, aldehydes or esters, anhydride, sulfhydryl or carboxyl groups such as derivatives of maleymil benzoic acid or of maleymil propionic acid and succinimidyl derivatives, groups derived from cyanogen bromide or chloride, carbonyldiimidazole, succinimide esters or sulfonyl halides.

11. The compound as claimed in claim 1, wherein the linkage between the analgesic molecule and the vector comprises at least one disulfide bridge.

12. The compound as claimed claim 1, wherein the analgesic molecule is morphine.

13. The compound as claimed in claim 12, wherein the vector is attached at the 6-position of said morphine molecule.

14. The compound as claimed in claim 1, wherein the analgesic molecule is morphine-6-glucuronide.

15. The compound as claimed in claim 14, wherein the vector is attached at the carboxylic acid of the glucuronide residue of said morphine-6-glucuronide molecule.

16. The use of the compound as claimed in claim 1, in a pharmaceutical composition, for preparing a medicinal product which is of use for treating pain.

17. The use as claimed in claim 16, wherein the pharmaceutical composition is in a form suitable for systemic, parenteral, oral, rectal, nasal, transdermal or pulmonary administration.
